(19) United States
(12) Patent Application Publication
(10) Pub. No.: US 2006/0206068 A1
(43) Pub. Date: Sep. 14, 2006

(54) Gauze on stick (GOS)

(76) Inventor: Richard G. Chavez, El Paso, TX (US)

Correspondence Address: Wilson Daniel Swayze, Jr., 3804 Clearwater Ct., Plano, TX, 75025, US

(21) Appl. No.: 11/391,190

(22) Filed: Mar. 28, 2006

Related U.S. Application Data
(63) Continuation-in-part of application No. 10/983,821, filed on Nov. 8, 2004.

(51) Int. Cl. A61M 35/00

(52) U.S. Cl. 604/290; 604/358; 602/42

(57) ABSTRACT

A device for the dispensing of gauzes and the like includes a housing device for the mechanical dispensing of multiple disposable gauzes, a cap at the end of the dispensing unit, and elements for the gauze to allow for sterility, grip and usefulness. The elements include a groove and stabilizing head system that allow for dispensing of the gauzes that are secured in place by a master circular disk and spring with a system of opposing saw tooth to allow forward motion of the gauze disk and the actual disposable gauze disk with an absorbent material on one side and on the other side a latex or a rubber belt to allow for the securing of gauze disk over the veni puncture site in an absorbent pad.

The present application is a continuation-in-part of the patent application Ser. No. 10/983,821 filed on Nov. 8, 2004.

### FIELD OF THE INVENTION

The field of invention or the technical field to which this invention pertains is medical devices for use in phlebotomy.

### BACKGROUND OF THE INVENTION

At this time, there is to my knowledge no related subject matter pertaining to this invention. If any description of related art could be made, then latex gloves or face shields used against biohazard contamination would fit this category. Yet, these devices are only one of a few that provide additional protection against accidental exposure with contaminated blood and needles. Each year, OSHA reports on the serious problems faced by health-care workers when dealing with drawing blood from patients in a safe and efficient manner and avoiding accidental needle sticks with potentially hazardous blood.

### SUMMARY OF THE INVENTION

The present invention is known herein as ‘Gauze On Stick’ abbreviated as GOS, and will greatly reduce the risk of acquiring a serious disease by health workers such as doctors, nurses and lab technicians by operating in the following manner: first, it will physically removed by approximately 3-4 inches the point of contact between a contaminated needle that is being withdrawn from a patient's vein and the health care's hand/arm (if used appropriately). This will be accomplished by eliminating the need to expose the health-care worker's hand to close proximity to the exit point of the needle while the needle is being drawn out and pressure is being applied with standard gauze. This is the precise moment when dangerous needle stick accidents can occur, caused by either the patient involuntarily jerking his arm forward and causing the health-care worker to stick himself/herself with a needle or by the health-care worker not paying attention when removing the needle from the vein of the patient.

The gauze on stick will include at a distal end a circular disk with a absorbent pad including cotton or another absorbent material that is removable and disposable that will be the collection vehicle of the oozing blood from a vein after a veni puncture. A veni puncture is known as a phlebotomy procedure in which blood is collected from a vein with a needle and syringe. The circular gauze disks will be preferably sterile units that will be manufactured in different sizes and colors. These gauze disks will be housed in the dispensing case or unit made of hard material, such as plastic. The circular shaped gauze disks include a top and bottom side. The top side includes the absorbent material and the bottom side may include two items; a latex strap that will serve if needed, as a means to secure the circular gauze disk over the veni puncture site and a small circular pad with an adhesive tape that will serve as a removable circular Band-Aid that can be placed over the veni puncture site.

Third, the gauze on stick will have a removable cap that protects the circular gauze disk when not in use. The absorbent material may be covered with a removable thin protective layer if a sterile environment is desired.

Fourth, the gauze disks may be stacked in bundles of 10-15 units and stored in the dispensing case or unit for their use and carrying. Advancement of the circular gauze disks will be accomplished by the constant pressure of the spring located in the last disc at the proximal end, or/and by the user by simply pressing down action at the proximal end of the dispensing unit. The dispensing case or unit may come in different sizes, colors, and possible exterior designs to be used on adult and pediatric patients.

The advantages of the gauze on stick are many. First, the gauze on stick provides the user with additional protection from accidental needle sticks. Second, it frees the user from having to hold manual hand pressure over a veni-puncture site by using the latex belt or strap secured to the circular gauze disk over the exit point of the needle and vein. Third, it provides a good apparatus to collect any blood leakage after the veni puncture has been accomplished.

The object of the gauze on stick is to decrease the incidence of needle sticks by health-care workers when collecting blood samples from patients in hospitals, clinics, homes, ambulances, labs and any other situation that warrants collecting blood samples.

### BRIEF DESCRIPTION OF THE DRAWINGS

The invention may be understood by reference to the following description taken in conjunction with the accompanying drawings, in which, like reference numerals identify like elements, and in which:

FIG. 1 illustrates a cross-sectional view of the dispensing unit of the present invention;

FIG. 2 illustrates the gauze disks of the present invention;

FIG. 3 illustrates an application of the present invention;

FIG. 4 illustrates a perspective view of the stacked gauze disks of the present invention

FIG. 5 illustrates the body and cap of the dispensing unit;

FIG. 6 illustrates a cross-sectional view of the dispensing unit and the stacked gauze disks;

FIG. 7 illustrates a cross-sectional view of the dispensing unit and the spring;

FIG. 8 illustrates a cross-sectional view of the dispensing unit with finger handles and clip;

FIG. 9 illustrates a cross-sectional view of a single disk;

FIG. 10 illustrates the disk of the present invention with a film;

FIG. 11 illustrates the disk of the present invention with a protective covering;

FIG. 12 illustrates the master disk without a spring.

### DETAILED DESCRIPTION

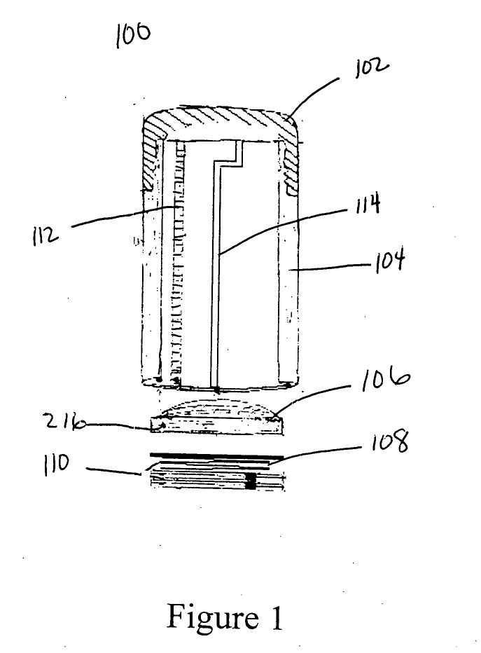

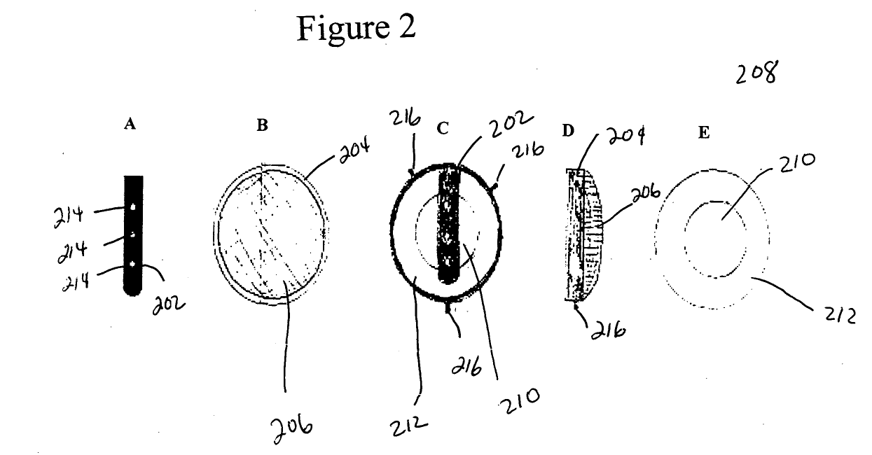

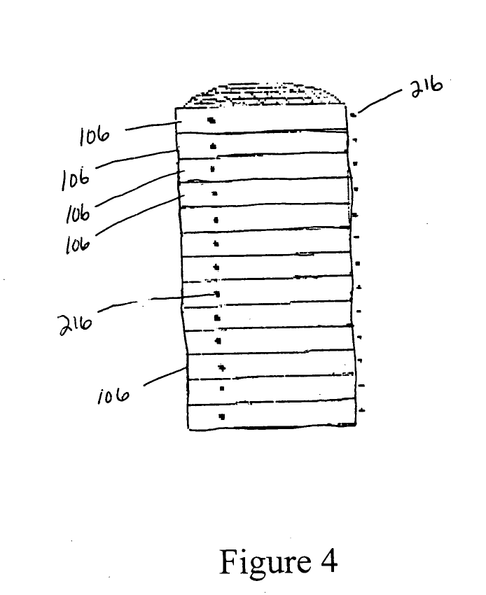

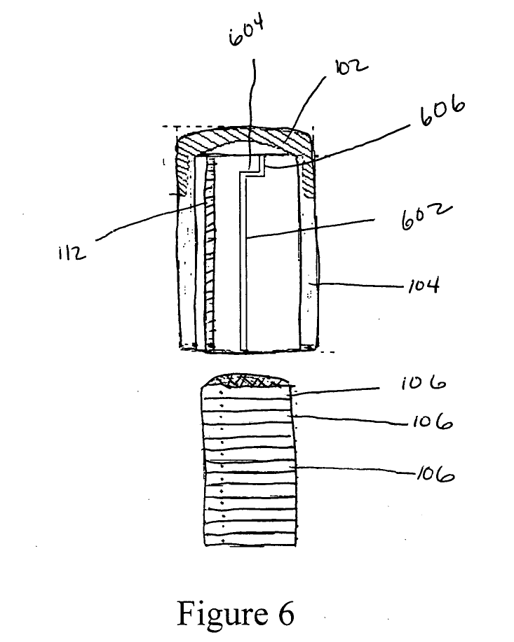

The gauze on stick is a relatively simple yet effective way in reducing the incidence of accidental needle sticks in the workplace from patients. A full description is as follows: it shall basically include two parts, the gauze disks 106 which may be circular in shape, oval, rectangular or any other suitable device in the dispensing case 100 or unit that houses the gauze disk 106 as shown in FIG. 1. The gauze disks 106 may include a thin covering over the gauze, the gauze itself 206, the disk 204 which may be plastic, the strap 202 which may be latex/rubber or a belt, and the band-aid 208 which may be circular as shown in FIG. 2. The top side of the disc includes the thin, removable plastic or paper sheath that covers the gauze 206 and ensures the sterile content. The gauze 206 may be made of cotton or another absorbent material with a hard supporting backing and disk 204 may be preferably made of hard plastic such as illustrated in FIG. 2. Attached to the bottom side of the disk includes a circular tape 212 and a circular absorbent pad 210 to serve as a band-aid 208. Also, the disk may include a strap 202 which may be a latex or rubber band anchored to the bottom side of the disk with small holes 214 formed in the middle that will serve to secure the strap 202 or band on various stabilizing head 216 on the disk as shown in FIG. 2. The gauze disks 106 may be stacked in 10-15 units, and these will be housed in the dispensing unit 100 as shown in FIG. 4 and in FIG. 6.

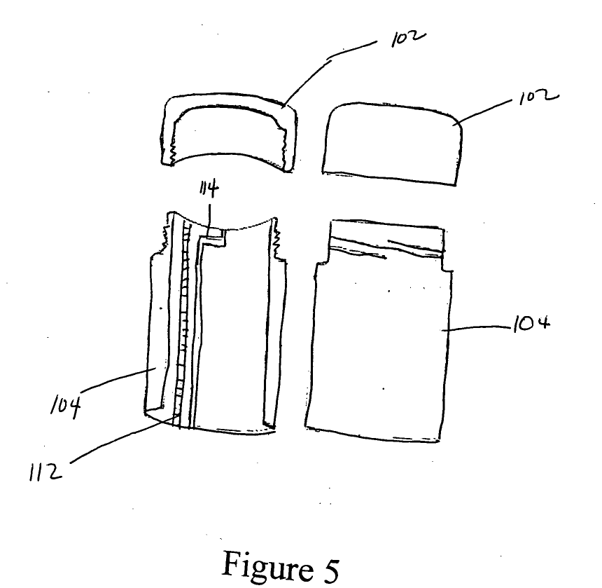

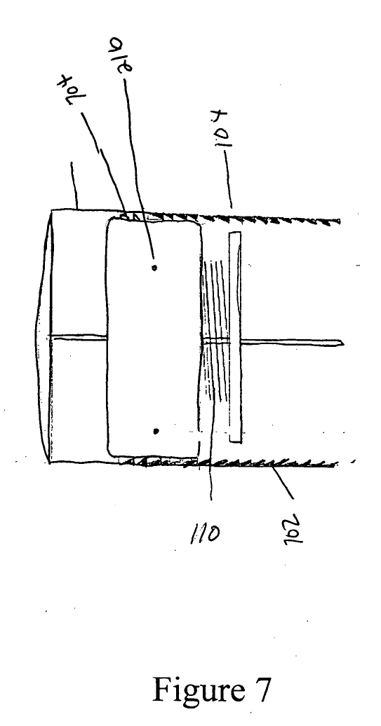

On the other hand, the dispensing unit 100 includes the dispensing unit body 104, the removable cap 102, and a master circular disk and spring 108 at the proximal end as shown in FIG. 1. The cap 102 is removed or replaced by screwing it on or off and serves to protect the gauze disks 106 that are housed within the dispensing unit 100 when not in use as shown in FIG. 5. Finally, the master circular disk 108 at the proximal end serves to secure and anchor the stack of 10-15 gauze disks 106 in the dispensing unit 100 as shown in FIGS. 1 and 7

The inner aspect of the dispenser may have a simple mechanism 112, 114 to guide and secure the stacked gauze disks 106 as shown in FIGS. 1 and 7.

The dispensing unit may come in two sizes, standard and small. The standard size will have the dimensions of but not limited to approximately: width 5 cm, length 10 cm, depth 5 cm as shown in FIG. 1. The small size will approximate the dimensions approximately: width 2.5 cm, length 10 cm in depth 2.5 cm. One material to manufacture the gauze on stick could be a hard, smooth disposable plastic of various colors.

The inner aspect of the dispenser 100 will have a simple mechanism 112, 114 to guide and secure the stacked gauze disk 106 as shown in FIGS. 1 and 7. It may have a system of grooves 114 or canals etched or formed inside the walls of the dispensing unit 100 that guide the stabilizing heads 216 of the circular gauze disk straight down the dispenser 100 for dispensing. There could be three guidance grooves 602, 604, 606 as illustrated in FIG. 6 or canals to guide the gauze disk 106.

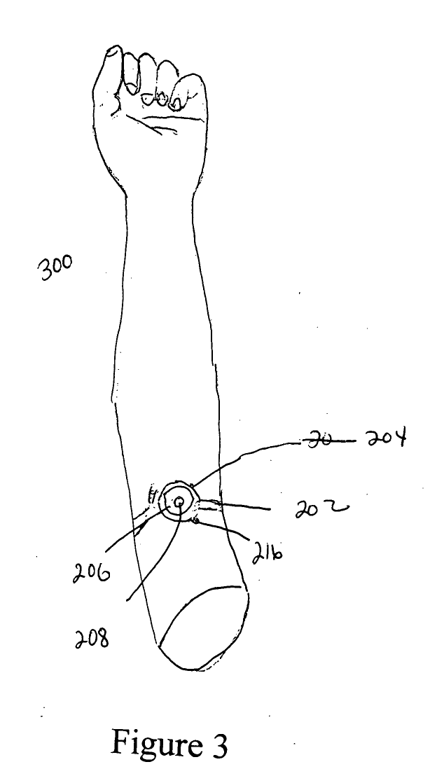

The inner aspect of the dispenser shall also have an anti-backward mechanism 112 to ensure that the gauze disks 106 move forward and not backward. This will be accomplished by having one or two or more rows of small saw tooth edges 702 pointing towards the dispensing point at the distal end. The master circular disk 108 at the end of the stack of gauze disks 106 may have two points that will engage the two rows of small saw tooth edges by having also small saw tooth edges 704 pointing the opposite direction towards the proximal end of the dispensing unit as shown in FIG. 7. The forward motion is needed to dispense the gauze disks 106 and is accomplished by the constant pressure of the spring 110 applied to the last disk 106 at the proximal end or by simply pressing down on the master disk 108 with the hand at the proximal end of the dispensing unit. The actual dispensing of the gauze disks 106 will be possible by pressing down on the gauze disk 106 and twisting it counterclockwise. This will be accomplished by the design of grooves 114 and the stabilizing head 216. By having a system of grooves 114 and stabilizing heads 216 that travels straight down in the longitudinal direction along the groove 602 and stop approximately 0.25 cm before reaching the end of the edge of the distal point of the dispensing unit, then turning and continuing approximately 1 cm along the groove 604 in the traverse direction then stopping and continuing approximately 0.25 cm straight down along the groove 606 again in the longitudinal direction until exiting the edge of dispensing unit 104 as shown in FIG. 6. Once the gauze disk 106 is dispensed, it could be secured over the venipuncture site with the strap 202 such as the latex or rubber band as shown in FIG. 3. The circular band-aid 208 could then be used, if needed, over the venipuncture site and a circular gauze disk 204 is discarded.

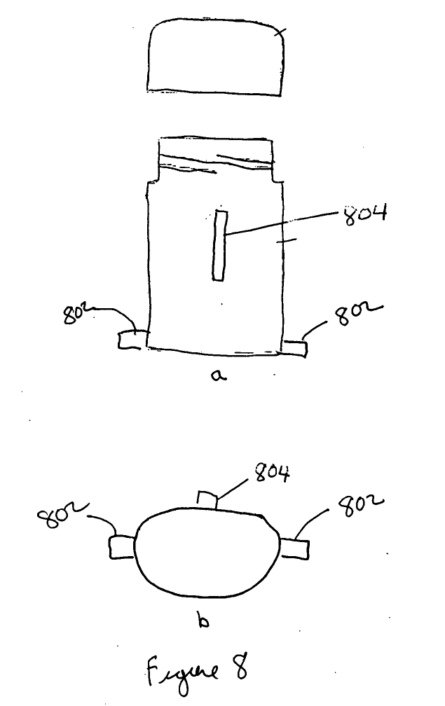

FIG. 8 a,b illustrates the dispensing unit with finger handles 802 shown positioned on the proximal end of the dispensing unit; however, the finger handles 802 could be located at other locations along the dispensing unit. FIG. 8 a,b additionally shows a pressure clip 804 attached to the dispensing unit. One end of the pressure clip 804 is connected to the dispensing unit, and the other end of the pressure clip 804 is not connected to the dispensing unit but allows the pressure clip 804 to be positioned over the edge of a pocket of the user so that the dispensing unit is conveniently carried.

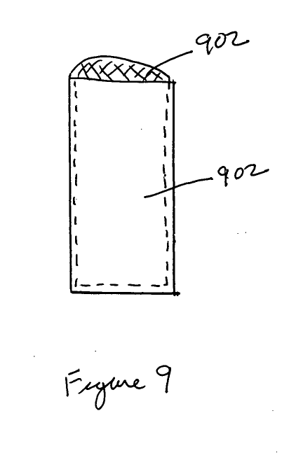

FIG. 9 illustrates a single disk being sized to substantially fill the dispensing unit to allow large amounts of blood to be absorbed by the gauze 902.

The dispensing unit may come in several sizes and colors.

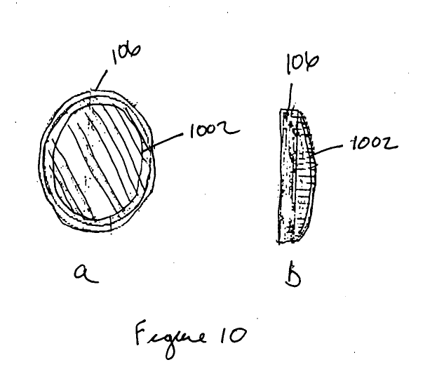

FIG. 10a in FIG. 10b illustrates the end view and side view respectively of the gauze disk 106 with a film 1002 to substantially cover the gauze. The film 1002 may include one of an anti-microbial film, or a quick coagulation film with gel style absorption.

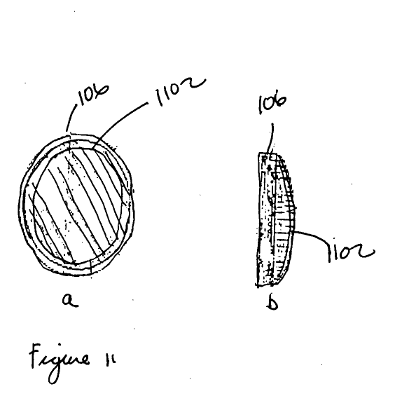

FIG. 11a and FIG. 11b illustrates the end view and side view respectively of the gauze disk 106 with a protective covering 1102 in order to maintain the sterility of the gauze disk 106.

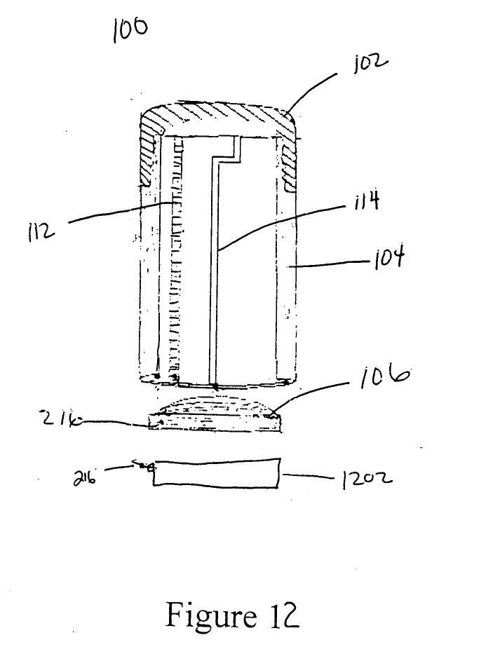

FIG. 12 illustrates a master disk 1202 without a spring.

This invention, the gauze on stick may be manufactured by a process known as injection molding. Each part should be manufactured in hard, durable, disposable plastic. The parts may be then assembled by machine or hand to include the soft parts such as the circular gauze, Band-Aid, and the latex/rubber band. The assembled gauze on stick could then be individually packaged in small plastic bags and irradiated if required to insure the sterile state of the finished product.

While the invention is susceptible to various modifications and alternative forms, specific embodiments thereof have been shown by way of example in the drawings and are herein described in detail. It should be understood, however, that the description herein of specific embodiments is not intended to limit the invention to the particular forms disclosed.

1. A dispenser, comprising:
a dispensing unit for dispensing a disk to be applied to a venipuncture;
said disk including an absorbing material to absorb blood from said venipuncture.

2. A dispenser as in claim 1, wherein said disk moves in only one direction in said dispensing unit.

3. A dispenser as in claim 1, wherein said dispensing unit includes an anti-backward mechanism to prevent said dispensing unit from moving backwards.

4. A dispenser as in claim 1, wherein said dispensing unit includes a first groove to guide said disk.

5. A dispenser as in claim 4, wherein said first groove is in a longitudinal direction of said dispensing unit.

6. A dispenser as in claim 4, wherein said dispensing unit includes a second groove in a traverse direction of said dispensing unit.

7. A dispenser as in claim 1, wherein said disk includes a spring to move said absorbing material.

8. A dispenser as in claim 1, wherein said disk includes a strap to be applied to an arm of a user.

9. A dispenser as in claim 4, wherein said disk includes a bead head to travel along said first groove.

10. A dispenser as in claim 8, wherein said disk includes a stabilizing head to engage said strap.

11. A dispenser as in claim 1, wherein said disk includes a Band-Aid.

12. A dispenser as in claim 1, wherein said absorbing material includes an anti-microbial film.

13. A dispenser as in claim 1, wherein said absorbing material includes a quick gel style absorption film.

14. A dispenser as in claim 1, wherein said absorbing material includes a protective covering to maintain sterility.

15. A dispenser as in claim 1, wherein said disk is moved by pressure from a user.

16. A dispenser as in claim 1, wherein said dispensing unit includes finger handles.

17. A dispensing unit as in claim 1, wherein said dispensing unit includes a clip.
